(19) United States
(12) Patent Application Publication
(10) Pub. No.: US 2017/0042526 A1
(43) Pub. Date: Feb. 16, 2017

(54) SELF-RETAINING RETRACTOR WITH INTEGRATED SUCTION AND LIGHT SOURCE

(71) Applicant: Robert TRIMARCHE, Lynchburg, MD (US)

(72) Inventor: Robert TRIMARCHE, Lynchburg, MD (US)

(21) Appl. No.: 15/306,237

(22) PCT Filed: Apr. 24, 2015
(86) PCT No.: PCT/US15/27448

§ 371 Date: Oct. 24, 2016

Related U.S. Application Data
(60) Provisional application No. 61/984,457, filed on Apr. 25, 2014.

(51) Int. Cl. A61B 17/02

(52) U.S. Cl. CPC A61B 17/0206; A61B 2217/005; A61B 90/30

(57) ABSTRACT

A retractor device according includes a first arm, a second arm connected to the first arm by a fastener, a first handle attached to a first end of the first arm, a second handle attached to a first end of the second arm, and a blade attached to each of a second end of the first arm and a second end of the second arm. A portion of the first arm includes a first hollow, the first hollow having a first opening and a second opening and a portion of the second arm includes a second hollow, the second hollow having a first opening and a second opening.

### CROSS-REFERENCE TO RELATED APPLICATION

This application claims priority from U.S. Provisional Application No. 61/984,457 filed Apr. 25, 2014 the disclosure of which is incorporated herein by reference.

### BACKGROUND

1. Field

Exemplary embodiments relate to surgical instruments, and more particularly, to tissue retractors for use in various surgical procedures.

2. Description of the Related Art

Conventional surgical retractors are shown in U.S. Patent Publication No. 2013/0281784, while convention suction tips are shown in U.S. Patent Publication No. 2011/0112372.

The importance of surgical retractors is well known in surgical procedures. Surgical retractors are very important because they determine the exposure of the operative field. A retractor is a surgical instrument that is used during operations to help surgeons maximize the field of view in the operative site by separating the edges of a surgical incision or wound and maintaining tissue and organs away from the area upon which the surgery is to be performed, while inflicting a minimum of trauma to the surrounding tissue. Surgical retractors are available in many sizes, shapes, and styles. Surgical retractors can be hand held or self-retaining dependent on the site of the body requiring the operation. The self-retaining retractor has a locking mechanism that allows hands free operation, thus allowing the surgeon to use both hands in the working surgical site. The blades of the retractors typically are at a right angle to the shaft The blades can be smooth, raked, or hooked. They can have removable or fixed blades. Common self-retaining retractors used in surgery include: Meyerding, Markham-Meyerding, Gelpi, Cerebellar Jansen, Weitlaner and Williams. These are all based on a solid, stainless steel frame with a self-locking, retaining mechanism.

Surgical retractors are non-hollow components made from materials that can be placed in an autoclave to be sterilized and so they may be reused for multiple, successive surgical procedures. A common material used in the making of retractors is stainless steel. Stainless steel is preferred because of its strength and its ability to be sterilized.

Surgical procedures, such as electro cauterization where destroying tissue using heat conduction from. a metal probe heated by electric current or to stop bleeding from small vessels, can generate surgical smoke. Surgical smoke can also include gases such as benzene, toluene, formaldehyde, and polycyclic aromatic hydrocarbons that are known carcinogens. These gases create an “acrid smell” in an operating room. Another danger from surgical smoke comes from the particle content of the smoke. Particulate smoke possesses serious health risks to surgical teams and is similar to second hand smoke from cigarettes. Another danger from surgical smoke is the transmission of diseases. The AIDS epidemic has focused attention on the routes by which HIV virus may be transmitted. One potential exposure route is inhalation of blood-containing “aerosols” infected with the virus in the operating room. The potential hazard of blood aerosol generated by electro surgery is from surgical tools capable of generating a wide distribution of particle sizes produced blood-containing particles in the breathable range. Surgical masks typically do not provide adequate respiratory protection against these aerosols produced in surgical smoke.

During surgery on soft tissues a surgeon would typically use an electro surgery device for cutting or dissecting, one or more retractors and one or more suction devices. The surgeon typically has a first assistant (e.g., other surgeons, physician assistants, nurse practitioners, surgical scrub nurses/technicians) to aid in the operation. The surgeon typically guards delicate tissue or has traction on the tissue edge being dissected with the use of suction device that is used to remove blood, fluids and tissue pieces; at the same time cutting with an electro surgery device that produces a surgical smoke plume. This usually requires the assistant to use a separate suction device held in the wound to evacuate the smoke.

An assistant surgeon performs continuous suctioning of surgical smoke, bone dust, bloody fluid and debris by placing a suction tube into the patient's surgical site and performs the necessary evacuation to aid in maintaining an optimal field of view for the operating surgeon. Every time the assistant performs the evacuation process, the surgeon may need to halt the procedure and stop for few moments while the evacuation process takes place. This may be detrimental the overall outcome of the procedure, due to prolonged anesthesia time and the assistant surgeon having additional tools in the delicate operative site thereby increasing the patient exposure to injury by possible human error.

When a wound is narrow, it is very awkward for an assistant to hold a retractor, a separate suction device that effectively evacuates the smoke and another for blood and irrigating fluids that does not get in the surgeon's line of site. In a deeper wound it can be difficult for the surgeon to get traction on the tissue due to the size of the surgeon's hands, the slippery surface of gloves on the one or more retractors or the bulk of sponges that are used to gain a friction hold on the surface of a slippery tissue. It is also more difficult to get adequate light to see the delicate tissues that need to be protected or extracted.

To overcome this type of problem, there are few surgical retractors that have been designed with integrated suction and external light source mounted on the tool to allow the surgeon to perform the evacuation process at the same time performing the surgical procedure. Although there are prior devices that combine suction and an external light source device, none have adequately been integrated into the standard self-retaining retraction devices. Advancements in surgical techniques and devices have included several devices for suction and retraction as well as “minimally invasive techniques”. However, these devices and techniques, while potentially reducing tissue trauma, do lend themselves to increased operating time and thus, increased exposure to anesthesia and infection increases.

However, exemplary embodiments overcomes the shortcomings and solves the problems associated with the prior devices that have been constructed. The exemplary embodiments provide an improved surgical field of view by combining continuous suctioning for evacuation of surgical smoke as well as an attachment of an external light source that can be mounted to the retractor blade so that it can be an integral part of a surgical retractor as a unitary piece.

The exemplary embodiment modify and improve several of the current self-retaining retractor systems by integrating suction and an external light source, thereby freeing the operator to perform the intricate tasks of surgery, with improved field of vision by reducing exposure to surgical smoke and improving the illumination of the surgical site. The type of retractors this modification is intended for includes, but is not limited to, Meyerding, Markham-Meyerding, Gelpi, Cerebellar Jansen, Weitlaner and Williams.

### SUMMARY

It is an aspect of the exemplary embodiments to provide a surgical retractor having an integral suction structure and light attachment which decreases surgery time, improves surgical performance, limits the exposure of surgeons to harmful pathogens, and minimizing the risk of injury to patients and surgeons.

A retractor device according to an exemplary embodiment includes a first arm, a second arm connected to the first arm by a fastener, a first handle attached to a first end of the first arm, a second handle attached to a first end of the second arm, and a blade attached to each of a second end of the first arm and a second end of the second arm. A portion of the first arm includes a first hollow, the first hollow having a first opening and a second opening and a portion of the second arm includes a second hollow, the second hollow having a first opening and a second opening.

According to an exemplary embodiment, a suction hose is removably attached to the second opening of the first hollow and the second opening of the second hollow.

According to an exemplary embodiment, each of the blades is at an end of the first and second arms opposite the first handle and the second handle.

According to an exemplary embodiment, the second opening of the first hollow and the second opening of the second hollow are at a position proximal to the fastener.

According to an exemplary embodiment, the first opening of the first hollow and the first opening of the second hollow are at a distal end of the first and second arms away from the fastener.

According to an exemplary embodiment, the first opening of the first hollow and the first opening of the second hollow are receive an object or a liquid from a target location.

According to an exemplary embodiment, the retractor includes a light attachment provided on the first and second arm configured to secure a light to the retractor.

According to an exemplary embodiment, the blades are one of a smooth blade, a hooked blade, or a hooked blade.

According to an exemplary embodiment, the blade of the first arm is a different type of blade as the blade of the second arm.

According to an exemplary embodiment, the fastener includes a screw-type fastener.

According to an exemplary embodiment, first openings are provided perpendicular to an extending direction of the first hollow and the second hollow, respectively.

According to an exemplary embodiment, the first openings are provided parallel to an extending direction of the first hollow and the second hollow, respectively.

According to an exemplary embodiment, the blades are removably connected to the first arm and the second arm.

A retractor device according to an exemplary embodiment includes a first arm, a second arm connected to the first arm by a fastener, a first handle attached to a first end of the first arm, a second handle attached to a first end of the second arm, a blade attached to each of a second end of the first arm and a second end of the second arm, and a light attachment provided on at least the first or the second arm configure to secure a light to the retractor.

### BRIEF DESCRIPTION OF THE DRAWINGS

These and/or other aspects will become apparent and more readily appreciated from the following description of exemplary embodiments, taken in conjunction with the accompanying drawings of which:

FIG. 1 is a perspective view showing a retractor according to an exempla , embodiment;

FIG. 2 is a cross-section view of the retractor arm along the line A-A according to an exemplary embodiment;

FIG. 3 is a cross-section view of the retractor arm along the line B-B according to an exemplary embodiment; and

FIG. 4 is a cross section view of a blade along the line C-C according to an exemplary embodiment.

### DETAILED DESCRIPTION

Reference will now be made in detail to exemplary embodiments, examples of which are illustrated in the accompanying drawings, wherein like reference numerals refer to like elements throughout. However, known functions associated with the exemplary embodiments or detailed descriptions on the configuration and other matters which would unnecessarily obscure the present disclosure will be omitted.

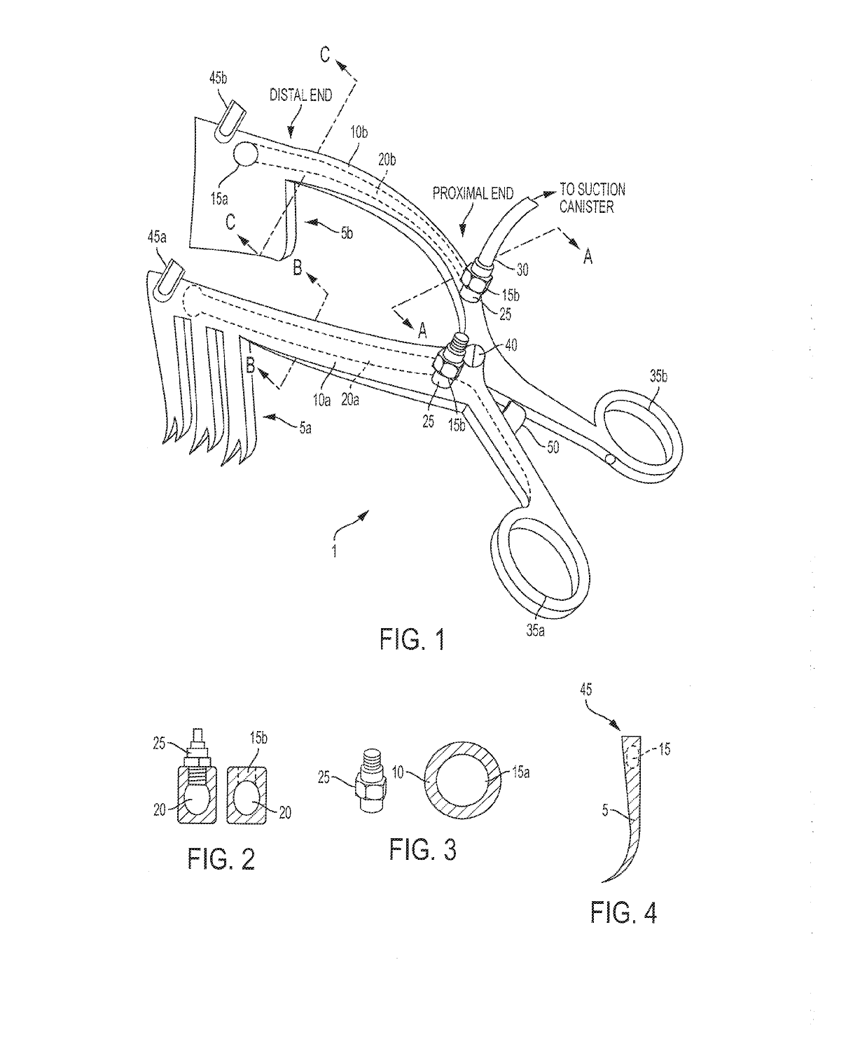

FIG. 1 is a perspective view of a retractor 1 according to an exemplary embodiment.

As shown in FIG. 1, the retractor 1 includes a first arm 10a and a second arm 10b are connected by a fastener 40. The first arm 10a and the second arm 10b are connected so that they can move with respect to each other. According to an exemplary embodiment, the fastener 40 is a screw-type fastener. However, exemplary embodiments are not limited thereto, and any fastening means known in the art may be employed which allows the first arm 10a and the second arm 10b to move with respect to each other.

FIG. 1 further shows that the first arm 10a and the second arm 10b have a first handle 35a and a second handle 35b attached at a first end thereof, respectively. The first handle 35a and the second handle 35b allow the user to manipulate the first arm 10a and the second arm 10b to move the arms 10a, 10b toward or away from each other. A lock 50 is provided on the first arm 35a and the second arm 35b, which enables the user to maintain the retractor I in a state where the arms 10a, 10b are at a distance from each other. In this manner, a cavity of the body may be accessed by holding structures of the body open to enable a surgeon to perform various techniques within a body cavity.

According to an exemplary embodiment, the first arm 10a includes a blade 5a and the second arm 10b includes a blade 5b. the blades 5a, 5b may be smooth, raked, or hooked, and may be fixed to the arms 10a, 10b or may be removably attached. The type of blade will depend on the technique being used and the area of the body on which a user is using the retractor 1.

As shown in FIG. 1, the first arm 10a and the second arm 10b each include a first hollow 20a and a second hollow 20b, respectively. The first hollow 20a and the second hollow 20b each include a first opening 15a and a second opening 15b. The first openings 15a are provided at an end of the retractor 1 distal to the handles 35a, 35b. In one preferred embodiment, the first openings 15a are provided at the blades 5a, 5b. However, the first openings 15a may be provided at any location along the arms 10a, 10b to remove liquid, debris, and other material from the surgical area. Further, although not shown, the retractor 1 may be provided with a plurality of openings along the arms 10a, 10b to provide additional locations to remove debris.

The location of the first openings 15a allows the surgeon to properly evacuate smoke, debris, liquids, and other objects from the surgical area without the need for additional tools. Further, the location of the first openings 15a on the blades 5a, 5b or the arms 10a, 10b eliminates the need for an assistant to provide a separate suction device, thereby reducing the time needed to perform the procedure and minimize the change of complicating factors when the surgeon has to move to perform suction.

As shown in FIG. 3, a cross section of the retractor 1 taken along the line B-B illustrates a first opening 15a according to an exemplary embodiment. The first opening 15a is provided in bath the first arm 10a and the second arm 10b. However, it will be understood that only one hollow 20a or 20b is provided. If a single hollow 20a or 20b is provided, then only one first opening 15a will be provided.

FIG. 4 is a cross-section taken along the line C-C of FIG. 1. As shown in FIG. 4, the blade 5 includes a first bole 15 and a light attachment 45.

At a second end of the first hollow 20a and the second hollow 20b are provided second openings 15b according to an exemplary embodiment. It will be understood that if only a single hollow 20a or 20b is provided, then only one second opening 15b will be provided.

The second openings 15b are provided on the first arm 10a and the second arm 10b at a location proximal to the handles 35a, 35b. As shown in FIG. 1, the second openings 15b include suction attachment ports 25. The suction attachment ports 25 are configured to be attached to the second opening 15b at a first end and are configured to be attached to a suction hoe 30 at a second end. The suction attachment ports 25 may be integrally formed with the second openings 15b or the suction attachment ports 25 may be separately formed and connected by any means known in the art, including but not limited to screw and thread, welding, and adhesives.

FIG. 2 is a cross section view along the line A-A showing the suction attachment port 25 connected to a second opening 15b according to an exemplary embodiment. As further shown in FIG. 2, the second. opening 15b is provided at one end of the hollow 20. In addition, FIG. 3 shows an exemplary embodiment of a suction attachment port 25. The suction attachment head 25 may be any structure known in the art capable of connecting to a suction hose 30, including but not limited to a hollow hex head hose nipple, as shown in FIG. 3.

A method of using the retractor 1 will now be explained.

According to an exemplary embodiment, a user places the blades 45a, 45b of the retractor 1 into an incision in a patient's body. Once the blades 45a, 45b are properly aligned, the physician manipulates the handles 35a, 35b to cause the arms 10a and 10b to separate. The lock 50 maintains the open position of the retractor 1.

The suction hose 30 may be attached to the suction attachment ports 25 at any time prior to or after placement of the retractor 1. Further, a light (not shown) may he placed in one or both of the light attachments 45a, 45b. The light attachments 45a, 45b allows light to be provided to the working area without the need for an assistant to hold the light in the location. This increases the visibility of the working area, which can decease the likelihood of mistakes, decrease surgery time, and therefore decrease patient recovery time and save money.

The location of the light attachments 45a, 45b allows the surgeon to properly view the surgical area without the need for additional tools. Further, the location of the light attachments 45a, 45b on the blades 5a, 5b or the arms 10a, 10b eliminates the need for an assistant to provide a separate lighting device, thereby reducing the time needed to perform the procedure and minimize the change of complicating factors associated with limited visibility of the surgical area.

During a procedure, the physician may be performing electro cauterization, producing surgical smoke, which can include gases such as benzene, toluene, formaldehyde, and polycyclic aromatic hydrocarbons that are known carcinogens. These gases also create an acrid smell in an operating room. Further, surgical smoke may increase the likelihood of contamination by making diseases airborne from the particle content of the smoke.

Thus, during surgery, the physician can turn the suction canister “on” (not shown), which is attached to the suction nose 30. Accordingly, smoke and other debris that might otherwise harm the physician and assistants is removed by the suction through the first opening 15a. This smoke and debris travels along hollows 20a, 20b to the second opening 15b. The smoke and debris pass through the suction attachment ports 25 and into the suction hose 30. The debris and smoke is subsequently removed to the suction canister and may be disposed of after surgery. The structure of the retractor according to an exemplary embodiment allows debris and smoke to he removed from the surgical area without the need for an assistant to hold a suction device in the surgical area. This increases the visibility of the working area, which can decease the likelihood of mistakes, decrease surgery time, and therefore decrease patient recovery time and save money.

According to an exemplary embodiment, the elements of the retractor I are formed from stainless steel. However, the retractor 1 may be formed of any material known in the art which is suitable for use in surgical procedures. Further, materials that are suitable for use in surgical procedures and can be cleaned by autoclaving or other similar techniques are preferable. In addition, the suction hose 30 may be made of any material suitable for use in surgical procedures, including but not limited to polymers used surgical techniques.

Although exemplary embodiments of the disclosure have been shown and described, it would be appreciated by those skilled in the art that changes may be made in these exemplary embodiments without departing from the principles and spirit of the exemplary embodiments, the scope of which is defined in the claims and their equivalents.

What is claimed is:

1. A retractor, comprising:
a first arm;
a second arm connected to the first arm by a fastener;
a first handle attached to a first end of the first arm;
a second handle attached to a first end of the second arm; and
a blade attached to each of a second end of the first arm and a second end of the second arm,
wherein a portion of the first arm includes a first hollow, the first hollow having a first opening and a second opening, and
wherein a portion of the second arm includes a second hollow, the second hollow having a first opening and a second opening.

2. The retractor of claim 1, wherein a suction hose is configured to be removably attached to the second opening of the first hollow and the second opening of the second hollow.

3. The retractor of claim 1, wherein each of the blades is at an end of the first and second arms opposite the first handle and the second handle.

4. The retractor of claim 1, wherein the second opening of the first hollow and the second opening of the second hollow are at a position proximal to the fastener.

5. The retractor of claim 1, wherein the first opening of the first hollow and the first opening of the second hollow are at a distal end of the first and second arms away from the fastener.

6. The retractor of claim 1, wherein the first opening of the first hollow and the first opening of the second hollow are configured to receive an object or a liquid, from a target location.

7. The retractor of claim 1, further comprising:
a light attachment provided on the first and second arm configured to secure a light to the retractor.

8. The retractor of claim 1, wherein the blades are one of a smooth blade, a hooked blade, or a hooked blade.

9. The retractor of claim 1, wherein the blade of the first arm is a different type of blade as the blade of the second arm.

10. The retractor of claim 1, wherein the fastener includes a screw-type fastener.

11. The retractor of claim 1, wherein the first openings are provided perpendicular to an extending direction of the first hollow and the second hollow, respectively.

12. The retractor of claim 1, wherein the first openings are provided parallel to an extending direction of the first hollow and the second hollow, respectively.

13. The retractor of claim 1, wherein the blades are removably connected to the first arm and the second arm.

14. A retractor, comprising:
a first arm;
a second arm connected to the first arm by a fastener;
a first handle attached to a first end of the first arm;
a second handle attached to a first end of the second arm;
a blade attached to each of a second end of the first arm and a second end of the second arm; and
a light attachment provided on at least the first or the second arm configure to secure a light to the retractor.
